(19) United States
(12) Patent Application Publication
(10) Pub. No.: US 2006/0129095 A1
(43) Pub. Date: Jun. 15, 2006

(54) Drug delivery apparatus

(76) Inventor: Leonard Pinchuk, Miami, FL (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/007,599

(22) Filed: Dec. 8, 2004

(51) Int. Cl. A61M 29/00

(52) U.S. Cl. 604/102.01; 604/537

(57) ABSTRACT

A drug delivery apparatus is provided which enables a medical professional to conveniently and controllably distribute drugs over an expanded target area within a patent. The apparatus comprises a catheter tube attached to a fluid delivery component. The catheter tube includes a distal end, a proximal end, a plurality of holes distributed along the length of the catheter tube, a first lumen which is not connected to any of said plurality of holes and extends from the proximal end toward the distal end of the tube, and a plurality of other lumens, each of which extends from the proximal end of the catheter tube to a respective hole or group of holes along the length of the tube and is in independent communication with a respective hole or group of holes. The fluid delivery component includes a first conduit connected to the first lumen at the proximal end of the catheter tube as well as a plurality of other conduits which are each connected to a respective other lumen at the proximal end of the catheter tube.

### FIELD OF THE INVENTION

The present invention relates to a new and improved apparatus for delivery of drugs to a patient. In the preferred embodiment, the drug delivery apparatus is in the form of a catheter.

### BACKGROUND INFORMATION

Catheters have long been used in the medical profession to inject or deliver drugs, fluids and other administrable media into a patient. Depending on its purpose, the media can be injected virtually anywhere into the patient such as into blood vessels, veins, arteries, organs, bile ducts, bronchials, and the like.

Catheters come in all shapes and sizes, and can be made from a variety of materials depending on its specific use. For example, catheters for injecting a drug into a blood vessel typically contain a small non-flexible metallic needle while catheters for delivery of a drug into the intestines through the rectum can be larger and made of flexible rubber or deformable plastic.

Most catheters contain a needle or a tube which includes a lumen that terminates at a hole at the distal end of the catheter. The drug is delivered through the lumen and to the hole at the distal end into the targeted area. As such, these types of catheter are best suited for very localized applications in which it is desired that the drug is delivered only to a specific site.

Other catheters have a variety of holes along the length of the catheter tube so that the drug or media is dispensed along the length of the tube as it travels through the lumen to the distal end of the catheter. A hole at the distal end of the catheter may or may not be present. These types of catheters are better suited for applications in which the targeted area is not isolated, and thus require distribution of the drug over an expanded or stretched area. For example, varicose vein conditions, which can affect veins along the span of the lower limbs, are best treated by distribution of sclerosing agents along the length of the veins. As such, a medical professional would opt to use catheters having holes along the length of the catheter tube to theoretically maximize the distribution of the sclerosing agent throughout the vein as opposed to using a catheter with only a hole at the distal end of the catheter tube. The holes along the catheter tube can be evenly distributed to theoretically achieve a more uniform dispensing of the sclerosing agent.

However, catheters frequently fail to perform as designed. For example, when the catheter as described in the preceding paragraph is inserted into the vein, certain areas of the vein may go into spasm and prevent the drug from exiting through some of the holes. Alternatively, some of the holes may become clogged. Once some of the holes are blocked, excess drug will be forced through the other holes, resulting in the unintended (and consequently uncontrolled) disproportionate distribution of the drug and/or the failure to deliver the drug to a specific area in the vein. Furthermore, for applications in which different amounts of drugs are needed over an expanded target area, there is no effective way for the medical professional to control the drug distribution.

To overcome this problem, other types of catheters, such as the Vein Rx catheter, employed two concentric catheter tubes. The inner catheter tube supports an occlusion balloon on its distal end, which when inflated stops blood flow in the vein/artery. The outer catheter tube, which is connected at the proximal end to a plunger and syringe, slides along the length of the inner catheter tube. Drug is delivered between the two catheter tubes by displacement of the plunger in the syringe and exits from the distal end of the outer catheter tube to the stretched target area as the outer catheter tube is slid across the inner catheter tube.

However, catheters of the type described in the preceding paragraph usually must be twice as long as the vein or target area in order to dispense the drug therein. For example, when treating a 32″ greater saphenous vein, the Vein Rx catheter is typically at least 64″ in length. This long length requires at least two medical professionals to operate the catheter, and raises practical and health concerns as well.

Thus, there is a need for a more simplified catheter which can be utilized in multiple applications, whether it is delivery of the drug or agent to a very specific site, or the distribution of the drug or agent over a stretched target area. For the latter application, there is also a need for a catheter which permits the medical professional to effectively control the amount of drug or agent to be delivered or injected to a specific part of the expanded target area.

### SUMMARY OF THE INVENTION

A new and improved drug delivery apparatus is provided.

In one embodiment, the drug delivery apparatus of the present invention comprises a catheter tube connected to a first conduit and a plurality of other conduits. The catheter tube includes a distal end, a proximal end, a plurality of holes distributed along the length of the catheter tube, a first lumen which is not connected to any of the plurality of holes and extends from the proximal end toward the distal end of the catheter tube, and a plurality of other lumens, each of which extends from the proximal end of the catheter tube to a respective hole or group of holes in the plurality of holes and each of which independently communicates with the respective hole or group of holes in the plurality of holes. The first conduit is connected to the first lumen at the proximal end of the catheter tube, and each conduit within the plurality of other conduits is connected to a respective lumen in the plurality of other lumens at the proximal end of the catheter tube.

In another embodiment of the present invention, the drug delivery apparatus comprises a catheter tube connected at the proximal end to a fluid delivery component. The catheter tube includes a distal end, a proximal end, a plurality of holes distributed along the length of the catheter tube, a first lumen which is not connected to any of the plurality of holes and extends from the proximal end toward the distal end of the catheter tube, and a plurality of other lumens, each of which extends from the proximal end of the catheter tube to a respective hole or group of holes in the plurality of holes and each of which independently communicates with the respective hole or group of holes in the plurality of holes. The fluid delivery component includes a housing, a first conduit connected to the first lumen, a plurality of other conduits, each of which is connected to a respective lumen in the plurality of other lumens, and a flange which mates with the housing and supports the first conduit and the plurality of other conduits.

In a further embodiment of the present invention, the drug delivery apparatus comprises a catheter tube connected at the proximal end to a fluid delivery component containing an identification component. The catheter tube includes a distal end, a proximal end, a plurality of holes distributed along the length of the catheter tube, a first lumen which is not connected to any of the plurality of holes and extends from the proximal end toward the distal end of the catheter tube, and a plurality of other lumens, each of which extends from the proximal end of the catheter tube to a respective hole or group of holes in said plurality of holes, and each of which independently communicates with the respective hole or group of holes in the plurality of holes. The fluid delivery component includes a housing, a first conduit connected to the first lumen, a plurality of other conduits, each of which is connected to a respective lumen in the plurality of other lumens, and an identification component which includes a passageway extending from the proximal end of the identification component to the distal end of the identification component. The passageway is in communication with a respective conduit from the plurality of other conduits at the distal end of the identification component and communicates with a different respective conduit in the plurality of other conduits upon adjustment of the identification component.

### BRIEF DESCRIPTION OF THE DRAWINGS

FIG. 1 is an illustration of an embodiment of the drug delivery apparatus in accordance with the present invention.

FIG. 2A is an illustration of another embodiment of the drug delivery apparatus in accordance with the present invention.

FIG. 2B is an illustration of the cross sectional view A-A of the embodiment shown in FIGS. 1 and 2A in accordance with the present invention.

FIG. 2C is an illustration of the cross-sectional view B-B of the embodiment shown in FIG. 2A in accordance with the present invention.

FIG. 3A is an illustration of another embodiment of the drug delivery apparatus in accordance with the present invention.

FIG. 3B is an illustration of the cross-sectional view C-C of the embodiment shown in FIG. 3A in accordance with the present invention.

FIG. 4 is an illustration of another embodiment of the drug delivery apparatus in accordance with the present invention.

FIG. 5A is an illustration of another embodiment of the drug delivery apparatus in accordance with the present invention.

FIG. 5B is an illustration of the cross sectional view D-D of the embodiment shown in FIG. 5A in accordance with the present invention.

FIG. 5C is an illustration of the cross-sectional view E-E of the embodiment shown in FIG. 5A in accordance with the present invention.

FIG. 6A is an illustration of another embodiment of the drug delivery apparatus in accordance with the present invention.

FIG. 6B is an illustration of the cross-sectional view F-F of the embodiment shown in FIG. 6A in accordance with the present invention.

FIG. 7 is an illustration of another embodiment of the drug delivery apparatus in accordance with the present invention.

FIG. 8A is a detailed illustration of a fluid delivery component embodiment of the apparatus shown in FIG. 7 in accordance with the present invention.

FIG. 8B is an expanded illustration of the area G of the fluid delivery component of FIG. 8A in accordance with the present invention.

### DETAILED DESCRIPTION OF CERTAIN PREFERRED EMBODIMENTS OF THE INVENTION

An apparatus is hereby disclosed which permits a medical professional to effectively control the amount of drugs delivered to a treatment area within a patient for multiple applications. The drug can be delivered through the apparatus using any known conventional techniques or equipment such as syringe injection, power pump, pressurized fluid flow, and the like. Drugs include any substance for which delivery to a site within a patient is desired, including but not limited to medication, diagnostic imaging agents, radiotherapeutic substances, therapeutic agents, fluids, minerals, saline, water, and the like.

As shown in FIG. 1, an embodiment of the drug delivery apparatus 1 comprises a catheter tube 6 connected to a first conduit 7 and a plurality of other conduits 8 (only one is shown in FIG. 1). The catheter tube 6 includes a distal end 9 (i.e., end farthest from the user), a proximal end 10 (i.e., end closest to the user), and a plurality of holes 2-5 distributed along the length of the catheter tube 6.

As shown in the Figures, it will be understood that the plurality of holes 2-5 are referenced for illustration purposes only, and is meant to include all holes present on catheter tube 6. The plurality of holes 2-5 can be arranged in any fashion along the length of the catheter tube 6, including but not limited to a helical, alternating, or random configuration. Holes 2-5 can be single holes or can be arranged into any number of groups, with each group containing the same or different number of holes. For example, as shown in FIG. 1, holes 2-5 can be configured in a helical arrangement in groups of three along the length of the catheter tube 6. Alternatively, holes 2-5 can be an alternating arrangement of single holes and groups of holes varying in number.

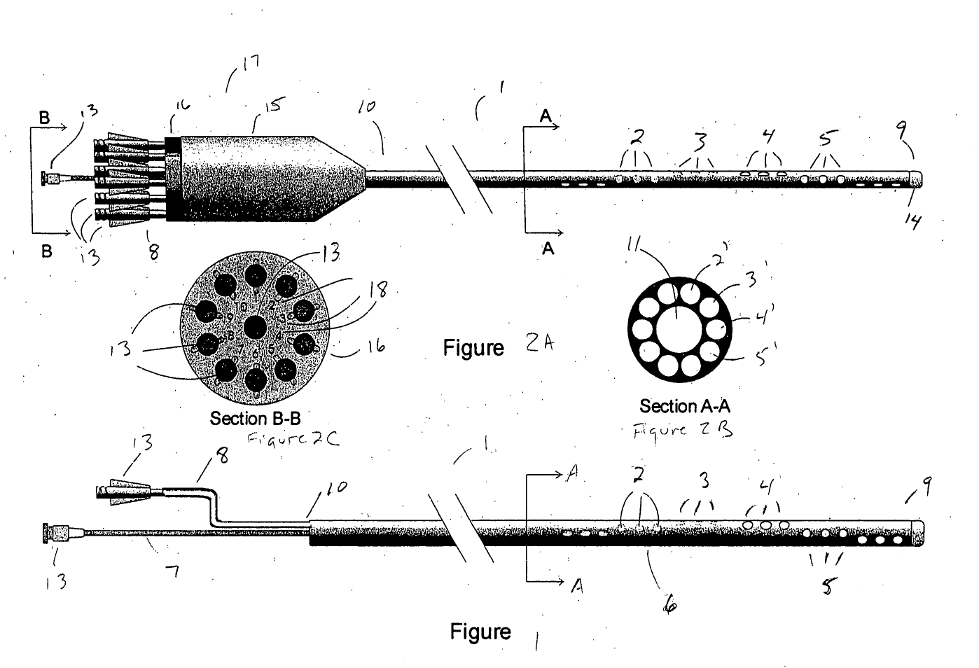

Catheter tube 6 further includes a first lumen 11 (shown in FIG. 2B) which is not connected to any of holes 2-5 and extends from the proximal end 10 toward the distal end 9 of catheter tube 6. Preferably, the first lumen 11 extends from the proximal end 10 to the distal end 9 of the catheter tube 6.

Catheter tube 6 also includes a plurality of other lumens 2′-5′, each of which extends from the proximal end 10 of the catheter tube 6 to a respective hole or group of holes in the plurality of holes 2-5. Furthermore, each of the individual lumens 2′-5′ independently communicates with a respective hole or group of holes in the plurality of holes 2-5. By way of example, lumen 2′ independently communicates with the group of holes 2 and extends from proximal end 10 of catheter tube 6 to the group of holes 2 such that any drug which enters lumen 2′ from proximal end 10 exits the catheter tube 6 exclusively at the holes 2. Similarly, lumen 3′ independently communicates with the group of holes 3 and extends from proximal end 10 of catheter tube 6 to the corresponding group of holes 3 such that any drug which enters lumen 3′ from proximal end 10 exits the catheter tube 6 exclusively at holes 3. And the same goes for the respective other lumens 4′-5′ with respect to corresponding holes 4-5 and henceforth. Each lumen of the plurality of other lumens 2′-5′ is said to be in independent communication with a respective hole or group of holes in the plurality of holes 2-5 since a drug can only exit a respective lumen at a respective hole or group of holes. As with holes 2-5, it will be understood that the plurality of other lumens 2′-5′ are referenced for illustration purposes only, and is meant to include all such other lumens present in catheter tube 6.

The plurality of other lumens 2′-5′ can be of any number depending on the number of individual holes or group of holes 2-5 for which independent communication is desired. For example, there can be a total of three lumens which comprise the plurality of other lumens 2′-5′: the first in independent communication with a single hole farthest from the distal end 9, the second in independent communication with a group of three holes in the middle of the catheter tube 6, and the third in communication with two holes closest to distal end 9.

Furthermore, the plurality of other lumens 2′-5′ can be configured using any type of arrangement. For example, FIG. 2B illustrates an annular arrangement for the plurality of other lumens 2′-5′. The plurality of other lumens 2′-5′ can also be arranged using an alternating, square, rectangular, or any other geometric design.

At the proximal end of catheter tube 6, each of the plurality of other lumens 2′-5′ is connected to and in communication with a respective conduit 8. Thus, any drug transmitted through a respective conduit 8 will enter a respective one of lumens 2′-5′ and exit a respective hole or group of holes 2-5.

Additionally, the first conduit 7 is connected to and in communication with first lumen 11 at the proximal end 10 of catheter tube 6. Consequently, if there is a hole (not shown) at the distal end 9 of catheter tube 6 which is in communication with first lumen 11, any drug transmitted through first conduit 7 will enter first lumen 11, and exit such distal end hole. On the other hand, if there is no distal end hole or if an atraumatic tip 14 is attached at the distal end 9, a guidewire or a stylet stiffening wire can be inserted into the first conduit 7, and thus first lumen 11, to permit some control of the movement of the catheter within the patient.

As shown in FIGS. 1 and 2A, optional connectors 13 can be attached to the first conduit 7 and plurality of other conduits 8 to facilitate the delivery of the drug or insertion of any other material (e.g., guidewire, etc.) into those conduits. Connectors 13 can be of any design and variety. For example, different sizes or types of Luer connectors can be attached to each of conduits 7 and 8 depending on its function, the kind of drug, or the mechanism used to deliver the drug to the conduits. Appropriate valves, check valves, injection septums or caps can be further be attached to the connectors 13 to prevent any possible backflow.

As shown in FIG. 2A, there can also be a housing 15 and a flange 16 which mates with the housing 15 and provides support for the first conduit 7 and the plurality of other conduits 8. In one embodiment, these components collectively form the fluid delivery component 17 of the drug delivery apparatus 1.

The housing 15 can be made from any material, be of any shape and size, and be permanently or removably attached to the catheter tube 6. The housing 15 can also serve as a handle by which the user can hold and transport the drug delivery apparatus.

As shown in FIGS. 2A and 2C, the first conduit 7 and the plurality of other conduits 8 extend through flange 16, which can also be made from any material, and be of any shape and size. The holes or gaps in flange 16 which conduits 8 extend through can be optionally identified with an indicator 18 such as numbers, letters, and the like, to indicate the relationship between a respective conduit 8, a respective lumen 2′-5′ and a respective hole or group of holes within the plurality of holes 2-5. For example, the indicator number “2” on flange 16 preferably indicates the respective conduit 8 which is attached to lumen 2′ which is in independent communication with holes 2. As such, any drugs injected through the respective connector 13 at indicator number “2” will exit at holes 2 on catheter tube 6, and so forth. Indicator 18 can be used to identify other relationships between any other conduits or holes as well.

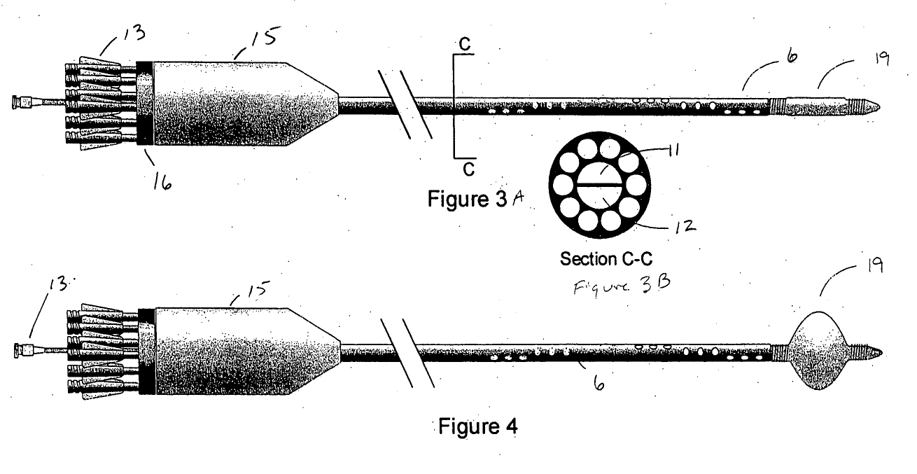

In another embodiment of the present invention shown in FIGS. 3A and 3B, the catheter tube 6 includes two lumens 11 and 12 which are not connected to any of the plurality of holes 2-5 and which extend from the proximal end 10 of the catheter tube 6 toward the distal end 9 of the catheter tube 6. Each of these lumens 11 and 12 can have its own independent function. For example, lumen 11 can be in independent communication with a hole or an occlusion balloon 19 at the distal end 9 of catheter tube 6. Thus, to stop blood flow through the vein or artery, inflation medium (gas or liquid) is injected through the first conduit 7 connected to lumen 11 until the occlusion balloon 19 is inflated (FIG. 4). A guidewire or stylet stiffening wire (not shown) can be inserted through another conduit (not shown) connected to the other lumen 12 to control the movement of the catheter. Additional lumens (and optionally conduits connected thereto) which are not connected to any of the plurality of holes 2-5 can be present as well.

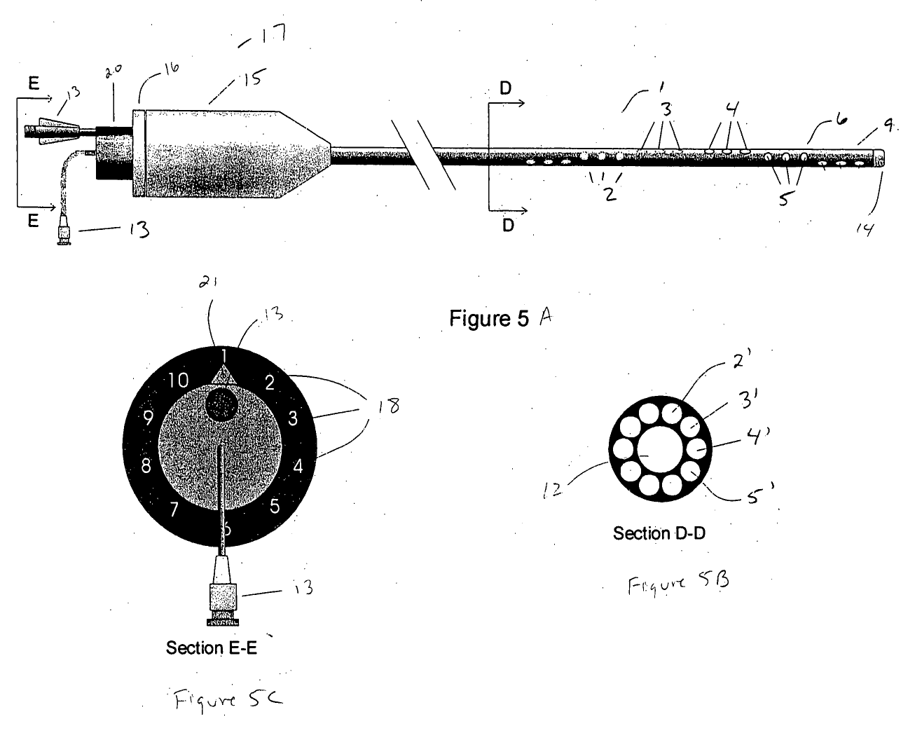

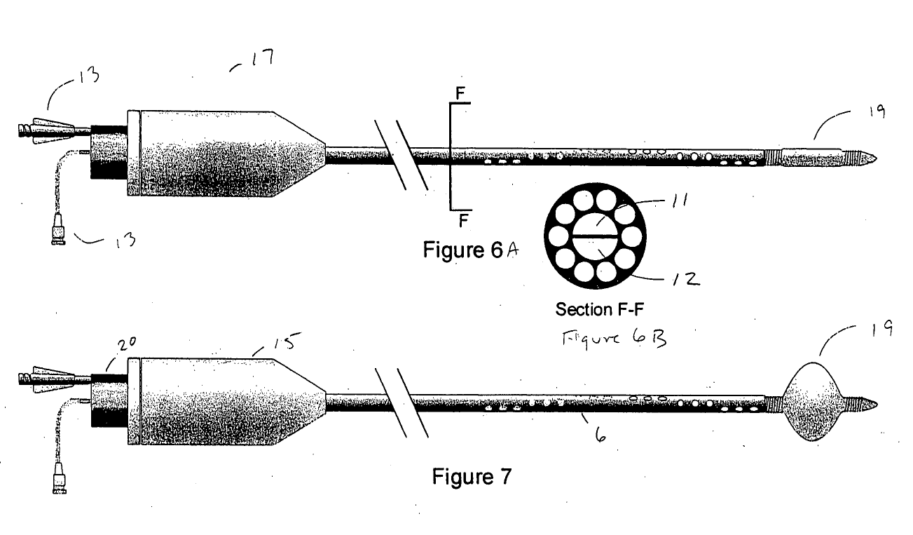

In yet another embodiment of the drug delivery apparatus 1 shown in FIGS. 5A-5C, 6-7 and 8A-8B, the fluid delivery component 17 includes a housing 15, a first conduit 7, a plurality of other conduits 8, and an identification component 20. An optional flange 16 capable of mating with housing 15 can be present as well.

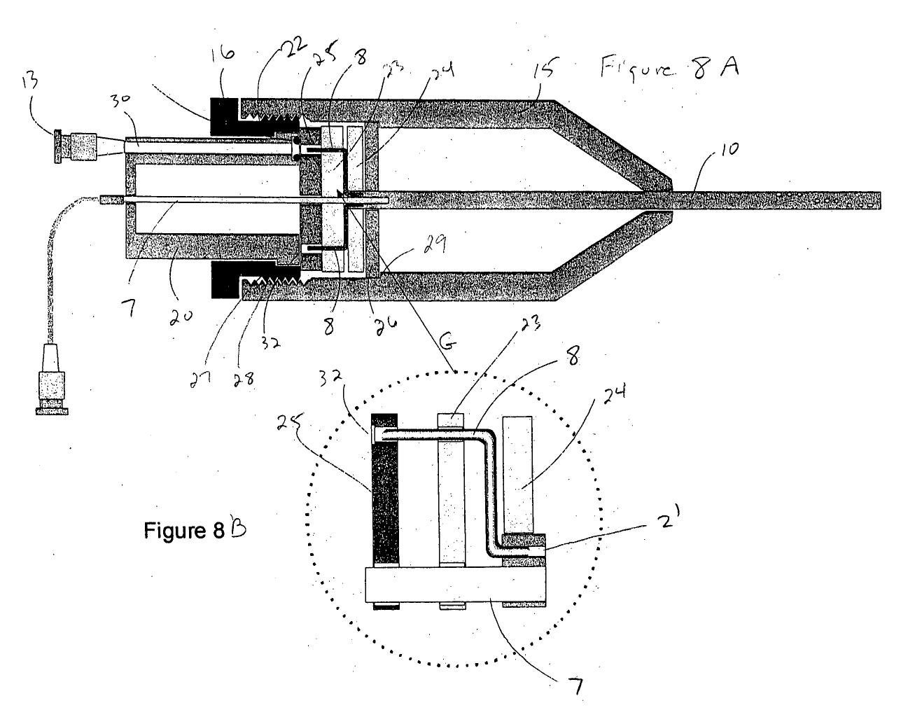

Within housing 15 at the proximal end 10 of catheter tube 6, the first conduit 7 is connected to the first lumen 11 while each of the other conduits 8 is connected to a respective lumen 2′-5′, which in turn, independently communicates with a respective hole or group of holes 2-5. The conduits 8 are supported by any conventional designs or techniques. For example, in an embodiment shown in FIGS. 8A and 8B, the conduits 8 are held in place by pads 23 and 24, which is sandwiched in between plates 25 and 26. Plate 26 can be fixed or held in place, for example by a change in internal housing dimensions or the presence of protrusion 29 within housing 15. In one embodiment, the mating or tightening of flange 16 with housing 15 via corresponding sets of threads 22 pushes plate 25 against pads 23, 24 and plate 26, thus securing the conduits 8 into place.

Additionally, the mating of flange 16 with housing 15 can also be used to secure the identification component 20 into place. For example, as shown in FIG. 8A, the tightening of flange 16 advances flange abutment 27 against the identification component ledge 28 and consequently pushes the identification component 20 into plate 25, thus securing the identification component 20 within housing 15.

As shown in FIG. 8A, the identification component 20 includes a passageway 30 which extends across the length of the identification component 20 (i.e., from the proximal end of the identification component to the distal end of the identification component). The passageway 30 can be a tube, lumen, or any other type of conduit permitting the movement of drugs from the distal end of the identification component 20 to the proximal end. A connector 13 can be attached to the distal end of the passageway 30 to adapt the passageway 30 to receive drugs from different delivery mechanisms (i.e., via syringe, pump, etc.) A check valve can be further attached to connector 13 to prevent backflow.

On the other side, the proximal end of the passageway 30 is intended to connect or communicate with a respective conduit 8. A thin well 32 can be present on plate 25 to serve as a detent to indicate when a respective conduit 8 is lined up with passageway 30. A gasket or O-ring 31 can be present at the proximal end of the passageway 30 to provide a more secure or sealed fluid communication between passageway 30 and a respectively selected conduit 8. The operation of passageway 30 will become clearer in the paragraphs that follow.

To use this drug delivery apparatus embodiment, the identification component 20 is adjusted (e.g., rotated, moved into position, etc.) to line passageway 30 up with a respective conduit 8 such that a drug delivered through passageway 30 will enter that respective conduit 8, into a respective lumen 2′-5′ and out of a respective hole or group of holes 2-5. To aid in determining which hole the drug will ultimately exit, each respective conduit 8 can be labeled by indicators 18 on flange 16. The identification component 20 would then be adjusted until the attached connector 13 and/or proximal end of the passageway 30 is lined up with a desired indicator 18.

For example, suppose the indicator number “2” identifies the respective conduit 8 which is connected to lumen 2′ which is in independent communication with holes 2. Thus, a medical professional wishing to deliver a drug to the target area around holes 2 would simply adjust the identification component 20 until the attached connector 13 is lined up with the indicator no. “2” before injecting the drug into the connector. The drug, once injected, travels through passageway 30, into the respective conduit 8, into lumen 2′ and out of holes 2. If the drug is then needed to be delivered to the target area around holes 5, then the medical professional would simply re-adjust the identification component 20 until the attached connector 13 is lined up with the indicator number “5” (assuming this identifies the respective conduit 8 which is connected to lumen 5′ which communicates independently with holes 5), re-inject the drug accordingly and henceforth.

To further aid the medical professional, the identification component 20 can also include a guide 21 (e.g., arrow, finger, marker, etc.) to confirm the lining up (and thus fluid communication) of connector 13 and the distal end of the passageway 30 with a respective conduit 8. Therefore, in the embodiment shown in FIGS. 5A-5C, where the identification component 20 is in the shape of a cylinder, the medical professional can rotate the identification component 20 clockwise or counterclockwise until the guide 21 “points” to the desired number indicator 18. For example, suppose the indicator number “2” identifies the respective conduit 8 which is connected to lumen 2′ which independently communicates with holes 2. Thus, a medical professional wishing to deliver a drug to the target area around holes 2 would simply turn the identification component 20 until the guide 21 points to the indicator number “2” before injecting the drug into the connector. The drug, once injected, travels through passageway 30, into the respective conduit 8, into lumen 2′ and out of holes 2. If the drug is then needed to be delivered to the target area around holes 5, the medical professional would turn the identification component 20 until the guide 21 points to the indicator number “5” (assuming this identifies the respective conduit 8 which is connected to lumen 5′ which communicates independently with holes 5), re-inject the drug accordingly and henceforth.

Thus, the fluid communication of the passageway 30 at the distal end of the identification component 20 with a respective conduit 8 can be changed to any of the other conduits 8 by the adjustment of identification component 20. The noted advantage of utilizing this type of an identification component 20 is that it permits the medical professional to use the same syringe or mechanism to deliver the drug through all of the holes 2-5 by simply adjusting the identification component 20 accordingly instead of having to use multiple syringes or mechanisms.

In an alternative embodiment, multiple passageways 30 can be present in identification component 20. For example, two passageways 30 can be present so that the medical professional can inject drugs concurrently into two respective conduits 8 without having to adjust the identification component 20.

### EXAMPLES

### Example 1

A patient is in need of treatment of varicose veins in the greater saphenous system. The varicosed saphenous vein is shown by Doppler ultrasound to extend from the saphenofemoral junction in the groin down to the ankle. An ultrasound is performed to determine the relative size of different portions of the vein. The saphenofemoral junction is marked on the outside of the leg with washable ink. A mark is placed at a site near the ankle where the catheter is to be introduced.

The patent lies down on a bed with his/her leg held in a horizontal position. The vein is filled with blood. The catheter as described in FIG. 2 is placed over the outside of the leg such that the distal tip of the catheter is located at the mark denoting the saphenofemoral junction. A mark is placed on the catheter (the mark may be a slidable O-ring located on the catheter) at the ankle mark.

An incision is made at the ankle mark. A catheter sheath introducer is optionally inserted at the ankle mark. All of the lumens of the catheter are purged of air with sodium tetradecyl sulfate (STS), a sclerosing agent commonly used to treat varicose veins. Check valves are connected to each of the connectors to purge the catheter and prevent backflow of blood into the catheter.

The catheter is inserted into the incision or through the catheter sheath introducer at the ankle site and maneuvered up the vein until the slidable O-ring marker on the catheter is just outside the introduction site. At this point, the tip of the catheter is at the saphenofemoral junction. The catheter is held in place by means of a suture or tape, or the like. The leg is elevated 30° thereby causing the blood to drain from the vein, therefore collapsing the vein. A tourniquet is optionally placed around the leg to ensure that there is no flow of blood in the leg.

While the vein is elevated and depleted of blood, sclerosing agent is injected sequentially into each of the connectors 13. In areas where the vein is larger, as determined by ultrasound, extra sclerosing agent is injected into the respective connectors 13 which correspond to the holes at those areas. Conversely, near the ankle, where the vein is smaller, less sclerosing agent is injected into the respective connector 13 which correspond to the holes at the ankle area. The injection is done sequentially among the connectors 13 so that the drug is delivered sequentially from the ankle area toward the groin area. The drug may be injected in reverse order as well, or in any alternating or random order as the medical professional sees fit.

The vein is optionally massaged to help circulate the sclerosing agent or to help force the drug into the tributaries. Once the entire length of the vein is sclerosed, the catheter is withdrawn and the wound closed. The leg is wrapped with compression bandages to maintain the collapsed state of the vein. After completion of the wrapping, the leg is lowered back to horizontal position and the patient is subsequently discharged.

### Example 2

Example 1 is repeated, except that the catheter as described in FIGS. 3 and 4 is used. The same procedure is followed, except that once the catheter is inserted into the vein, the occlusion balloon 19 is inflated with sterile saline to ensure that there is no blood flow in the vein. Sclerosing is performed with the balloon 19 inflated as described in Example 1. At the end of the procedure, the balloon is deflated and the catheter is removed.

Specific details of several embodiments of the invention have been set forth in order to provide a thorough understanding of the present invention. It will be apparent to one skilled in the art that other embodiments can be used and changes made without departing from the scope of the present invention. Furthermore, well known features that can be provided through the level of skill in the art have been omitted or streamlined for the purpose of simplicity in order to facilitate understanding of the present invention.

What is claimed is:

1. A drug delivery apparatus comprising:
a catheter tube which includes
a distal end,
a proximal end,
a plurality of holes distributed along the length of the catheter tube,
a first lumen which is not connected to any of said plurality of holes and extends from the proximal end toward the distal end of the catheter tube, and
a plurality of other lumens, each of which extends from the proximal end of the catheter tube to a respective hole or group of holes in said plurality of holes, and each of which independently communicates with said respective hole or group of holes in said plurality of holes,
a first conduit connected to said first lumen at the proximal end of said catheter tube, and
a plurality of other conduits, each of which is connected to a respective lumen in said plurality of other lumens at the proximal end of the catheter tube.

2. The drug delivery apparatus of claim 1, wherein the plurality of holes are arranged in groups.

3. The drug delivery apparatus of claim 1, wherein the plurality of holes are arranged helically.

4. The drug delivery apparatus of claim 1, wherein the plurality of other lumens are arranged in an annular configuration.

5. The drug delivery apparatus of claim 1, wherein said first lumen extends from the proximal end of the catheter tube to the distal end of the catheter tube.

6. The drug delivery apparatus of claim 1, wherein said catheter tube includes at least two lumens which are not connected to any of said plurality of holes, and which extend from the proximal end of the catheter tube toward the distal end of the catheter tube.

7. The drug delivery apparatus of claim 1, comprising an atraumatic tip attached to the distal end of the catheter tube.

8. The drug delivery apparatus of claim 1, comprising an inflatable balloon attached to the distal end of the catheter tube.

9. The drug delivery apparatus of claim 1, comprising an opening at the distal end of the catheter tube.

10. The drug delivery apparatus of claim 1, comprising connectors attached to said first conduit and each of said plurality of other conduits.

11. The drug delivery apparatus of claim 1, comprising
a housing and
a flange which mates with said housing and supports said first conduit and said plurality of other conduits.

12. The drug delivery apparatus of claim 11 wherein said flange include indicators which identify the communication relationship between each of said plurality of other conduits and said respective hole or groups of holes in said plurality of holes.

13. A drug delivery apparatus comprising:
a catheter tube which includes
a distal end,
a proximal end,
a plurality of holes distributed along the length of the catheter tube,
a first lumen which is not connected to any of said plurality of holes and extends from the proximal end toward the distal end of the catheter tube, and
a plurality of other lumens, each of which extends from the proximal end of the catheter tube to a respective hole or group of holes in said plurality of holes, and each of which independently communicates with said respective hole or group of holes in said plurality of holes, and
a fluid delivery component at the proximal end of the catheter tube, which includes
a housing,
a first conduit connected to said first lumen,
a plurality of other conduits, each of which is connected to a respective lumen in said plurality of other lumens, and
a flange which mates with said housing and supports said first conduit and said plurality of other conduits.

14. The drug delivery apparatus of claim 13, wherein the plurality of other lumens are arranged in an annular configuration.

15. The drug delivery apparatus of claim 13, wherein the plurality of holes are arranged helically.

16. The drug delivery apparatus of claim 13, wherein said catheter tube includes at least two lumens which are not connected to any of said plurality of holes, and which extend from the proximal end of the catheter tube toward the distal end of the catheter tube.

17. The drug delivery apparatus of claim 13, comprising connectors attached to said first conduit and each of said plurality of other conduits.

18. The drug delivery apparatus of claim 13, wherein said flange include indicators which identify the communication relationship between each of said plurality of other conduits and said respective hole or groups of holes in said plurality of holes.

19. A drug delivery apparatus comprising:
a catheter tube which includes
a distal end,
a proximal end,
a plurality of holes distributed along the length of the catheter tube,
a first lumen which is not connected to any of said plurality of holes, and extends from the proximal end toward the distal end of the catheter tube, and
a plurality of other lumens, each of which extends from the proximal end of the catheter tube to a respective hole or group of holes in said plurality of holes, and each of which independently communicates with said respective hole or group of holes in said plurality of holes, and
a fluid delivery component at the proximal end of the catheter tube, which includes
a housing,
a first conduit connected to said first lumen,
a plurality of other conduits, each of which is connected to a respective lumen in said plurality of other lumens, and
an identification component including a passageway which extends from the proximal end of said identification component to the distal end of said identification component where said passageway is in communication with a respective conduit in said plurality of other conduits,
wherein the adjustment of said identification causes said passageway to be in communication with a different respective conduit in said plurality of other conduits.

20. The drug delivery apparatus of claim 19, comprising a flange capable of mating with said housing, wherein the mating of said flange with said housing secures the identification component within said housing.

21. The drug delivery apparatus of claim 19, wherein the plurality of holes are arranged in groups.

22. The drug delivery apparatus of claim 19, wherein the plurality of holes are arranged helically.

23. The drug delivery apparatus of claim 19, wherein the plurality of other lumens are arranged in an annular configuration.

24. The drug delivery apparatus of claim 19, wherein said first lumen extends from the proximal end of the catheter tube to the distal end of the catheter tube.

25. The drug delivery apparatus of claim 19, wherein said catheter tube includes at least two lumens which are not connected to any of said plurality of holes, and which extend from the proximal end of the catheter tube toward the distal end of the catheter tube.

26. The drug delivery apparatus of claim 19, comprising an atraumatic tip attached to the distal end of the catheter tube.

27. The drug delivery apparatus of claim 19, comprising an inflatable balloon attached to the distal end of the catheter tube.

28. The drug delivery apparatus of claim 19, comprising an opening at the distal end of the catheter tube.

29. The drug delivery apparatus of claim 19, comprising a connector attached to said passageway at the proximal end of said identification component.

30. The drug delivery apparatus of claim 20, wherein said flange include indicators which identify the communication relationship between each of said plurality of other conduits and said respective hole or groups of holes in said plurality of holes.

31. The drug delivery apparatus of claim 30, wherein said identification component includes a guide which confirms the communication between said passageway and a respective conduit in said plurality of other conduits.

32. The drug delivery apparatus of claim 19, wherein said identification component includes at least two passageways that extend from the proximal end of said identification component to the distal end of said identification component, each passageway being in independent communication with a respective conduit in said plurality of other conduits.

33. The drug delivery apparatus of claim 19, wherein said adjustment of identification component includes rotating the identification component in a clockwise or counterclockwise direction.
